(19) United States
(12) Patent Application Publication
(10) Pub. No.: US 2011/0092742 A1
(43) Pub. Date: Apr. 21, 2011

(54) Process for Producing Alkylbenzene Hydroperoxides

(76) Inventors: Jihad M. Dakka, Whitehouse Station, NJ (US); Stephen Zushma, Clinton, NJ (US)

(21) Appl. No.: 12/996,238

(22) PCT Filed: Jul. 14, 2009
(86) PCT No.: PCT/US09/50481

§ 371 Date: Dec. 3, 2010

Related U.S. Application Data
(60) Provisional application No. 61/091,850, filed on Aug. 26, 2008.

(51) Int. Cl. C07C 407/00; C07C 37/08

(52) U.S. Cl. 568/573; 568/798

(57) ABSTRACT

In a process for producing alkylbenzene hydroperoxides, a feed comprising (i) sec-butylbenzene, (ii) cumene in an amount greater than 10 wt % of the total feed and (iii) at least one of iso-butylbenzene and tert-butylbenzene in an amount up to 20 wt % of the total feed is contacted with an oxygen-containing gas in the presence of a catalyst comprising a cyclic imide of the general formula (I):

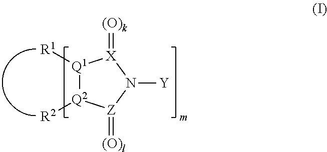

wherein each of R¹ and R² is independently selected from hydrocarbyl and substituted hydrocarbyl radicals having 1 to 20 carbon atoms, or from the groups SO₃H, NH₂, OH, and NO₂ or from the atoms H, F, Cl, Br, and I, provided that R¹ and R² can be linked to one another via a covalent bond; each of Q¹ and Q² is independently selected from C, CH, N and CR³; each of X and Z is independently selected from C, S, CH₂, N, P and elements of Group 4 of the Periodic Table; Y is O or OH; k is 0, 1, or 2; l is 0, 1, or 2; m is 1 to 3; and R³ can be any of the entities listed for R¹. The contacting is conducted under conditions to convert the sec-butylbenzene and cumene to their associated hydroperoxides.

### CROSS REFERENCE TO RELATED APPLICATION

This application claims the benefit of prior U.S. provisional application Ser. No. 61/091,850 filed Aug. 26, 2008, which is hereby incorporated by reference in its entirety.

### FIELD

The present invention relates to a process for producing alkylbenzene hydroperoxides and optionally for converting the resultant hydroperoxides into phenol.

### BACKGROUND

Phenol is an important product in the chemical industry. For example, phenol is useful in the production of phenolic resins, bisphenol A, ε-caprolactam, adipic acid, alkyl phenols, and plasticizers.

Currently, the most common route for the production of phenol is the Hock process. This is a three-step process involving alkylation of benzene with propylene to produce cumene, followed by oxidation of the cumene to the corresponding hydroperoxide and then cleavage of the hydroperoxide to produce equimolar amounts of phenol and acetone. However, the world demand for phenol is growing more rapidly than that for acetone. In addition, the cost of propylene relative to that for butenes is likely to increase, due to a developing shortage of propylene. Thus, a process that uses butenes instead of or as well as propylene as feed and co-produces methyl ethyl ketone may be an attractive alternative route to the production of phenol.

It is known that phenol and methyl ethyl ketone can be co-produced by a variation of the Hock process in which sec-butylbenzene is oxidized to obtain sec-butylbenzene hydroperoxide and the hydroperoxide is decomposed to the desired phenol and methyl ethyl ketone. An overview of such a process is described in pages 113-122 and 261-263 of Process Economics Report No. 22B entitled “Phenol”, published by the Stanford Research Institute in December 1977.

It is also known that a mixture of phenol with varying quantities of methyl ethyl ketone and acetone can be produced by oxidizing a feed containing cumene and sec-butylbenzene and then cleaving the resultant hydroperoxides. By controlling the weight ratio of cumene to sec-butylbenzene in the feed, the ratio of acetone to methyl ethyl ketone in the product can be varied depending on market conditions. See European Published Application No. 1,088,809 and U.S. Pat. No. 7,282,613.

However, the production of phenol using sec-butylbenzene as the or one of the alkylbenzene precursors is accompanied by certain problems which either are not present or are less severe with a cumene-based process. For example, in comparison to cumene, oxidation of sec-butylbenzene to the corresponding hydroperoxide is very slow in the absence of a catalyst and is very sensitive to the presence of impurities. As a result, U.S. Pat. Nos. 6,720,462 and 6,852,893 have proposed the use of cyclic imides, such as N-hydroxyphthalimide, as catalysts to facilitate the oxidation of alkylbenzenes, such as sec-butylbenzene.

Sec-butylbenzene can be produced by alkylating benzene with n-butenes over an acid catalyst. The chemistry is very similar to ethylbenzene and cumene production. However, as the carbon number of the alkylating agent increases, the number of product isomers also increases. For example, ethylbenzene has one isomer, and propylbenzene has two isomers (cumene and n-propylbenzene), but butylbenzene has four isomers (n-, iso-, sec-, and t-butylbenzene). These by-products, especially iso-butylbenzene and t-butylbenzene, have boiling points very close to sec-butylbenzene and hence are difficult to separate from sec-butylbenzene by distillation (see table below).

| Butylbenzene | Boiling Point, ° C. |
|---|---|
| t-Butylbenzene | 169 |
| i-Butylbenzene | 171 |
| s-Butylbenzene | 173 |
| n-Butylbenzene | 183 |

In addition, although sec-butylbenzene production in the benzene alkylation step can be maximized by using a pure n-butene feed, in practice it is desirable to employ more economical butene feeds, such as Raffinate-2. A typical Raffinate-2 contains 0-1% butadiene and 0-5% isobutene. With this increased isobutene in the feed, a higher production of iso-butylbenzene and t-butylbenzene is inevitable. However, isobutylbenzene and tert-butylbenzene are known to be inhibitors to the oxidation of sec-butylbenzene to the corresponding hydroperoxide. In the past, this has been a significant disincentive to the use of the Hock process to produce phenol from sec-butylbenzene.

In our U.S. Published Patent Application No. 2007/0265476, published Nov. 17, 2007, we have shown that when an alkylbenzene feedstock of the formula:

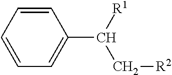

in which R¹ and R² each independently represent an alkyl group having from 1 to 4 carbon atoms, such as sec-butylbenzene, is oxidized in the presence of a cyclic imide catalyst, such as N-hydroxyphthalimide, the rate of oxidation is substantially unaffected by the presence of iso-butylbenzene and tert-butylbenzene impurities even at levels as high as 3 wt % of the sec-butylbenzene. Although Application No. 2007/0265476 indicates that the alkylbenzene feedstock may also contain cumene, it teaches that the cumene should only be present in an amount that does not exceed 10%, preferably that does not exceed 8%, and more preferably that does not exceed 5%, of the feedstock. Moreover, no information is provided in Application No. 2007/0265476 as to the affect of the presence of cumene on the sec-butylbenzene oxidation step.

According to the present invention, it has now unexpectedly been found that, when a mixture of cumene and sec-butylbenzene is oxidized in the presence of a cyclic imide catalyst, such as N-hydroxyphthalimide, the inclusion of small quantities, up to 20 wt %, of iso-butylbenzene and/or tert-butylbenzene significantly improves both the rate of conversion of the cumene and sec-butylbenzene and the selectivity to the desired hydroperoxides.

### SUMMARY

In one aspect, the invention resides in a process for producing alkylbenzene hydroperoxides, the process comprising contacting a feed comprising (i) sec-butylbenzene, (ii) cumene in an amount greater than 10 wt % of the total feed and (iii) at least one of iso-butylbenzene and tert-butylbenzene in an amount up to 20 wt % of the total feed with an oxygen-containing gas in the presence of a catalyst comprising a cyclic imide of the general formula (I):

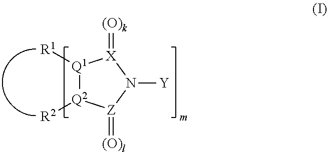

wherein each of R¹ and R² is independently selected from hydrocarbyl and substituted hydrocarbyl radicals having 1 to 20 carbon atoms, or from the groups SO₃H, NH₂, OH, and NO₂ or from the atoms H, F, Cl, Br, and I, provided that R¹ and R² can be linked to one another via a covalent bond;
each of Q¹ and Q² is independently selected from C, CH, N and CR³;
each of X and Z is independently selected from C, S, CH₂, N, P and elements of Group 4 of the Periodic Table;

### Y is O or OH;

k is 0, 1, or 2;
l is 0, 1, or 2;
m is 1 to 3; and
R³ can be any of the entities listed for R¹, and wherein said contacting is conducted under conditions to convert said sec-butylbenzene and cumene to the associated hydroperoxides.

In one embodiment, said cyclic imide obeys the general formula (II):

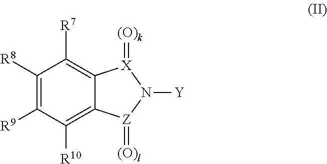

wherein each of R⁷, R⁸, R⁹, and R¹⁰ is independently selected from hydrocarbyl and substituted hydrocarbyl radicals having 1 to 20 carbon atoms, or from the groups SO₃H, NH₂, OH, and NO₂ or from the atoms H, F, Cl, Br, and I,
each of X and Z is independently selected from C, S, CH₂, N, P and elements of Group 4 of the Periodic Table;

### Y is O or OH;

k is 0, 1, or 2; and
l is 0, 1, or 2.

Conveniently, said cyclic imide comprises N-hydroxyphthalimide.

Conveniently, said feed comprises from about 1 wt % to about 15 wt %, such as from about 1 wt % to about 10 wt %, of iso-butylbenzene and/or tert-butylbenzene. When both isomers are present, the wt % ranges are based on the combined weight of the two isomers.

Conveniently, said feed comprises from about 15 wt % to about 50 wt % of cumene.

Conveniently, said contacting is conducted at a temperature of between about 90° C. and about 150° C., such as between about 100° C. and about 140° C., for example between about 115° C. and about 130° C. Typically, said contacting is conducted at a pressure between about 15 kPa and about 500 kPa, such as at a pressure between about 15 kPa and about 150 kPa.

Conveniently, said cyclic imide is present in an amount between about 0.05 wt % and about 5 wt %, such as between about 0.1 wt % and about 1 wt %, of the sec-butylbenzene and cumene in said feed during said contacting.

Typically, the process further comprises cleaving the hydroperoxides produced by said contacting to produce phenol, acetone and methyl ethyl ketone.

Conveniently, the cleaving is conducted in the presence of a catalyst. In one embodiment, the cleaving is conducted in the presence of a homogeneous catalyst, such as at least one of sulfuric acid, perchloric acid, phosphoric acid, hydrochloric acid, p-toluenesulfonic acid, ferric chloride, boron trifluoride, sulfur dioxide and sulfur trioxide. In another embodiment, the cleaving is conducted in the presence of a heterogeneous catalyst, such as smectite clay.

Conveniently, the cleaving is conducted at a temperature of about 40° C. to about 120° C. and/or a pressure of about 100 to about 1000 kPa and/or a liquid hourly space velocity (LHSV) based on the hydroperoxides of about 1 to about 50 hr⁻¹.

In one embodiment, the process further comprises converting the phenol produced by the cleaving to bisphenol A.

### BRIEF DESCRIPTION OF THE DRAWINGS

FIG. 1 is a graph comparing the cumene conversion against time on stream (TOS) for the uncatalyzed air oxidation of a mixed cumene and sec-butylbenzene feed containing 0 wt %, 5 wt % and 20 wt % of tert-butylbenzene (TBB).

FIG. 2 is a graph comparing the cumene hydroperoxide (CHP) selectivity against cumene conversion for the uncatalyzed air oxidation of a mixed cumene and sec-butylbenzene feed containing 0 wt %, 5 wt % and 20 wt % of tert-butylbenzene.

FIG. 3 is a graph comparing the sec-butylbenzene (SBB) conversion against time on stream for the uncatalyzed air oxidation of a mixed cumene and sec-butylbenzene feed containing 0 wt %, 5 wt % and 20 wt % of tert-butylbenzene.

FIG. 4 is a graph comparing the sec-butylbenzene hydroperoxide (SBBHP) selectivity against sec-butylbenzene conversion for the uncatalyzed air oxidation of a mixed cumene and sec-butylbenzene feed containing 0 wt %, 5 wt % and 20 wt % of tert-butylbenzene.

FIG. 5 is a graph comparing the cumene conversion against time on stream for the uncatalyzed air oxidation of a mixed cumene and sec-butylbenzene feed containing 0 wt %, 5 wt % and 20 wt % of iso-butylbenzene (iso BB).

FIG. 6 is a graph comparing the cumene hydroperoxide selectivity against cumene conversion for the uncatalyzed air oxidation of a mixed cumene and sec-butylbenzene feed containing 0 wt %, 5 wt % and 20 wt % of iso-butylbenzene.

FIG. 7 is a graph comparing the sec-butylbenzene conversion against time on stream for the uncatalyzed air oxidation of a mixed cumene and sec-butylbenzene feed containing 0 wt %, 5 wt % and 20 wt % of iso-butylbenzene.

FIG. 8 is a graph comparing the sec-butylbenzene hydroperoxide selectivity against sec-butylbenzene conversion for the uncatalyzed air oxidation of a mixed cumene and sec-butylbenzene feed containing 0 wt %, 5 wt % and 20 wt % of iso-butylbenzene.

FIG. 9 is a graph comparing the cumene conversion against time on stream for the air oxidation of a mixed cumene and sec-butylbenzene feed both with (w) and without (wo) 0.1 wt % of N-hydroxyphthalimide (NHPI) and with and without 5 wt % of tert-butylbenzene.

FIG. 10 is a graph comparing the cumene hydroperoxide selectivity against cumene conversion for the air oxidation of a mixed cumene and sec-butylbenzene feed both with and without 0.1 wt % of N-hydroxyphthalimide and with and without 5 wt % of tert-butylbenzene.

FIG. 11 is a graph comparing the sec-butylbenzene conversion against time on stream for the air oxidation of a mixed cumene and sec-butylbenzene feed both with and without 0.1 wt % of N-hydroxyphthalimide and with and without 5 wt % of tert-butylbenzene.

FIG. 12 is a graph comparing the sec-butylbenzene hydroperoxide selectivity against sec-butylbenzene conversion for the air oxidation of a mixed cumene and sec-butylbenzene feed both with and without 0.1 wt % of N-hydroxyphthalimide and with and without 5 wt % of tert-butylbenzene.

FIG. 13 is a graph comparing the sec-butylbenzene conversion against time on stream for the air oxidation of a mixed cumene and sec-butylbenzene feed both with and without 0.1 wt % of N-hydroxyphthalimide and with and without 5 wt % of iso-butylbenzene.

FIG. 14 is a graph comparing the sec-butylbenzene hydroperoxide selectivity against sec-butylbenzene conversion for the air oxidation of a mixed cumene and sec-butylbenzene feed both with and without 0.1 wt % of N-hydroxyphthalimide and with and without 5 wt % of iso-butylbenzene.

FIG. 15 is a graph comparing the cumene conversion against time on stream for the air oxidation of a mixed cumene and sec-butylbenzene feed both with and without 0.1 wt % of N-hydroxyphthalimide and with and without 5 wt % of iso-butylbenzene.

FIG. 16 is a graph comparing the cumene hydroperoxide selectivity against cumene conversion for the air oxidation of a mixed cumene and sec-butylbenzene feed both with and without 0.1 wt % of N-hydroxyphthalimide and with and without 5 wt % of iso-butylbenzene.

### DETAILED DESCRIPTION OF THE EMBODIMENTS

Described herein is a process for oxidizing a mixture of cumene and sec-butylbenzene into the corresponding hydroperoxides and optionally for converting the resultant hydroperoxides into phenol. The process employs a cyclic imide as the oxidation catalyst and is based on the unexpected finding that, with the catalytic oxidation of such a mixed feed, the inclusion of small quantities, up to 20 wt %, of iso-butylbenzene and/or tert-butylbenzene significantly improves both the rate of conversion of the cumene and sec-butylbenzene and the selectivity to the desired hydroperoxides.

### Alkylbenzene Feedstock

The alkylbenzene feedstock employed in the present process comprises a mixture of sec-butylbenzene with cumene in an amount greater than 10 wt % of the total feedstock and at least one of iso-butylbenzene and tert-butylbenzene in an amount up to 20 wt % of the total feedstock. The maximum of 20 wt % is applied to the combined amount of iso-butylbenzene and tert-butylbenzene when both are present. More typically, the feedstock contains from about 15 wt % to about 50 wt %, such as from about 20 wt % to about 40 wt % of cumene and from about 1 wt % to about 15 wt %, such as from about 1 wt % to about 10 wt %, of iso-butylbenzene and/or tert-butylbenzene, with the remainder being sec-butylbenzene.

The alkylbenzene feedstock can be produced by alkylating benzene with a mixture of a C₃ alkylating agent and a C₄ alkylating agent, with the amount of the C₃ alkylating agent being controlled so as to generate the required amount of cumene in the alkylbenzene product. Alternatively, the cumene and butylbenzene components in the feedstock can be produced in separate alkylation operations and then mixed in the requisite proportions to produce the desired feedstock composition.

Irrespective of whether the cumene and butylbenzene components are produced simultaneously or sequentially, any C₃ compound capable of substituting a propyl group for a benzene hydrogen atom can be used as the C₃ alkylating agent. Thus, for example, the C₃ alkylating agent can comprise one or more of a propyl halide, a propyl alcohol and propylene. Generally, the C₃ alkylating agent comprises propylene.

Similarly, the C₄ alkylating agent can comprise one or more butyl halides, butyl alcohols and/or C₄ olefins. Generally, the C₄ alkylating agent comprises at least one linear butene, namely butene-1, butene-2 or a mixture thereof. However, since the alkylbenzene feedstock employed in the present process comprises iso-butylbenzene and/or tert-butylbenzene in addition to sec-butylbenzene, the C₄ alkylating agent normally also comprises at least some iso-butene. This is an advantage of the present process, since most commercially available C₄ olefin streams contain a mixture of linear butenes and iso-butene. For example, the following C₄ hydrocarbon mixtures are generally available in any refinery employing steam cracking to produce olefins; a crude steam cracked butene stream, Raffinate-1 (the product remaining after solvent extraction or hydrogenation to remove butadiene from the crude steam cracked butene stream) and Raffinate-2 (the product remaining after removal of butadiene and isobutene from the crude steam cracked butene stream). Generally, these streams have compositions within the weight ranges indicated in Table 1 below.

TABLE 1

| | | Raffinate 1 | | Raffinate 2 | |
|---|---|---|---|---|---|
| | Crude C₄ | Solvent | Hydro- | Solvent | Hydro- |
| Component | stream | Extraction | genation | Extraction | genation |
| Butadiene | 30-85% | 0-2% | 0-2% | 0-1% | 0-1% |
| C4 | 0-15% | 0-0.5% | 0-0.5% | 0-0.5% | 0-0.5% |
| acetylenes | | | | | |
| Butene-1 | 1-30% | 20-50% | 50-95% | 25-75% | 75-95% |
| Butene-2 | 1-15% | 10-30% | 0-20% | 15-40% | 0-20% |
| Isobutene | 0-30% | 0-55% | 0-35% | 0-5% | 0-5% |
| N-butane | 0-10% | 0-55% | 0-10% | 0-55% | 0-10% |
| Iso-butane | 0-1% | 0-1% | 0-1% | 0-2% | 0-2% |

Other refinery mixed C₄ streams, such as those obtained by catalytic cracking of naphthas and other refinery feedstocks, typically have the following composition:

| | |
|---|---|
| Propylene | 0-2 wt % |
| Propane | 0-2 wt % |
| Butadiene | 0-5 wt % |
| Butene-1 | 5-20 wt % |
| Butene-2 | - 10-50 wt % |
| Isobutene | 5-25 wt % |
| Iso-butane | 10-45 wt % |
| N-butane | 5-25 wt % |

C₄ hydrocarbon fractions obtained from the conversion of oxygenates, such as methanol, to lower olefins more typically have the following composition:

| | |
|---|---|
| Propylene | 0-1 wt % |
| Propane | 0-0.5 wt % |
| Butadiene | 0-1 wt % |
| Butene-1 | 10-40 wt % |
| Butene-2 | 50-85 wt % |
| Isobutene | 0-10 wt % |
| N- + iso-butane | 0-10 wt % |

Any one or any mixture of the above C₄ hydrocarbon mixtures can be used as a C₄ alkylating agent in the present process. In some cases, however, it may be advantageous to subject these mixtures to one or more pretreatment steps to remove butadiene and/or reduce the isobutene level prior to alkylation. For example, butadiene can be removed by extraction or selective hydrogenation to butene-1, whereas the isobutene level can be reduced by selective dimerization or reaction with methanol to produce MTBE. Conveniently, the C₄ alkylating agent employed in the present process contains from about 5 wt % to about >0.5 wt % iso-butene and less than 0.1 wt % butadiene.

In addition to other hydrocarbon components, commercial C₃ and C₄ hydrocarbon mixtures typically contain other impurities which could be detrimental to the alkylation process. For example, refinery C₃ and C₄ hydrocarbon streams typically contain nitrogen and sulfur impurities, whereas C₃ and C₄ hydrocarbon streams obtained by oxygenate conversion processes typically contain unreacted oxygenates and water. Thus, prior to the alkylation step, these mixtures may also be subjected to one or more of sulfur removal, nitrogen removal and oxygenate removal, in addition to butadiene removal and isobutene removal. Removal of sulfur, nitrogen, oxygenate impurities is conveniently effected by one or a combination of caustic treatment, water washing, distillation, adsorption using molecular sieves and/or membrane separation. Water is also typically removed by adsorption.

Conveniently, the feed to the or each alkylation step of the present process contains less than 1000 ppm, such as less than 500 ppm, for example less than 100 ppm, water and/or less than 100 ppm, such as less than 30 ppm, for example less than 3 ppm, sulfur and/or less than 10 ppm, such as less than 1 ppm, for example less than 0.1 ppm, nitrogen.

Irrespective of whether the C₃ and C₄ alkylation steps are conducted simultaneously or sequentially, the alkylation catalyst used in the or each alkylation step is conveniently a crystalline molecular sieve of the MCM-22 family. The term “MCM-22 family material” (or “material of the MCM-22 family” or “molecular sieve of the MCM-22 family” or “MCM-22 family zeolite”), as used herein, includes one or more of:
molecular sieves made from a common first degree crystalline building block unit cell, which unit cell has the MWW framework topology. (A unit cell is a spatial arrangement of atoms which if tiled in three-dimensional space describes the crystal structure. Such crystal structures are discussed in the “Atlas of Zeolite Framework Types”, Fifth edition, 2001, the entire content of which is incorporated as reference);
molecular sieves made from a common second degree building block, being a 2-dimensional tiling of such MWW framework topology unit cells, forming a monolayer of one unit cell thickness, preferably one c-unit cell thickness;
molecular sieves made from common second degree building blocks, being layers of one or more than one unit cell thickness, wherein the layer of more than one unit cell thickness is made from stacking, packing, or binding at least two monolayers of one unit cell thickness. The stacking of such second degree building blocks can be in a regular fashion, an irregular fashion, a random fashion, or any combination thereof; and
molecular sieves made by any regular or random 2-dimensional or 3-dimensional combination of unit cells having the MWW framework topology.

Molecular sieves of the MCM-22 family include those molecular sieves having an X-ray diffraction pattern including d-spacing maxima at 12.4±0.25, 6.9±0.15, 3.57±0.07 and 3.42±0.07 Angstrom. The X-ray diffraction data used to characterize the material are obtained by standard techniques using the K-alpha doublet of copper as incident radiation and a diffractometer equipped with a scintillation counter and associated computer as the collection system.

Materials of the MCM-22 family include MCM-22 (described in U.S. Pat. No. 4,954,325), PSH-3 (described in U.S. Pat. No. 4,439,409), SSZ-25 (described in U.S. Pat. No. 4,826,667), ERB-1 (described in European Patent No. 0293032), ITQ-1 (described in U.S. Pat. No. 6,077,498), ITQ-2 (described in International Patent Publication No. WO97/17290), MCM-36 (described in U.S. Pat. No. 5,250,277), MCM-49 (described in U.S. Pat. No. 5,236,575), MCM-56 (described in U.S. Pat. No. 5,362,697), UZM-8 (described in U.S. Pat. No. 6,756,030), and mixtures thereof. Molecular sieves of the MCM-22 family are preferred as the alkylation catalyst since they have been found to be highly selective to the production of sec-butylbenzene, as compared with the other butylbenzene isomers. Preferably, the molecular sieve is selected from (a) MCM-49, (b) MCM-56 and (c) isotypes of MCM-49 and MCM-56, such as ITQ-2.

The alkylation catalyst can include the molecular sieve in unbound or self-bound form or, alternatively, the molecular sieve can be combined in a conventional manner with an oxide binder, such as alumina, such that the final alkylation catalyst contains for example between 2 and 80 wt % sieve.

In one embodiment, the catalyst is unbound and has a crush strength much superior to that of catalysts formulated with binders. Such a catalyst is conveniently prepared by a vapor phase crystallization process, in particular a vapor phase crystallization process that prevents caustic used in the synthesis mixture from remaining in the zeolite crystals as vapor phase crystallization occurs.

Prior to use in the alkylation process, the MCM-22 family zeolite, either in bound or unbound form, may be contacted with water, either in liquid or vapor form, under conditions to improve its sec-butylbenzene selectivity. Although the conditions of the water contacting are not closely controlled, improvement in sec-butylbenzene selectivity can generally be achieved by contacting the zeolite with water at temperature of at least 0° C., such as from about 10° C. to about 50° C., preferably for a time of at least 0.5 hour, for example for a time of about 2 hours to about 24 hours. Typically, the water contacting is conducted so as to increase the weight of the catalyst by 30 to 75 wt % based on the initial weight of the zeolite.

The alkylation conditions employed depend on whether the cumene and butylbenzenes are produced in a single alkylation process or in separate processes. However, in either case, the conditions conveniently include a temperature of from about 60° C. to about 260° C., for example between about 100° C. and about 200° C. and/or a pressure of 7000 kPa or less, for example from about 1000 to about 3500 kPa and/or a weight hourly space velocity (WHSV) based on C₃ and/or C₄alkylating agent of between about 0.1 and about 50 hr⁻¹, for example between about 1 and about 10 hr⁻¹ and/or a molar ratio of benzene to alkylating agent of from about 1 to about 20, preferably about 3 to about 10, more preferably about 4 to about 9.

The reactants can be in either the vapor phase or partially or completely in the liquid phase and can be neat, i.e., free from intentional admixture or dilution with other material, or they can be brought into contact with the zeolite catalyst composition with the aid of carrier gases or diluents such as, for example, hydrogen or nitrogen. Preferably, the reactants are at least partially in the liquid phase.

Although the alkylation step is highly selective towards monoalkylbenzene(s), the effluent from the alkylation reaction will normally contain some polyalkylated products, as well as unreacted aromatic feed and the desired monoalkylated species. The unreacted aromatic feed is normally recovered by distillation and recycled to the alkylation reactor. The bottoms from the benzene distillation are further distilled to separate monoalkylated product from any polyalkylated products and other heavies. Depending on the amount of polyalkylated products present in the alkylation reaction effluent, it may be desirable to transalkylate the polyalkylated products with additional benzene to maximize the production of the desired monoalkylated species.

Transalkylation with additional benzene is typically effected in a transalkylation reactor, separate from the alkylation reactor, over a suitable transalkylation catalyst, such as a molecular sieve of the MCM-22 family, zeolite beta, MCM-68 (see U.S. Pat. No. 6,014,018), zeolite Y or mordenite. The transalkylation reaction is typically conducted under at least partial liquid phase conditions, which suitably include a temperature of 100 to 300° C. and/or a pressure of 1000 to 7000 kPa and/or a weight hourly space velocity of 1 to 50 hr⁻¹ on total feed and/or a benzene/polyalkylated benzene weight ratio of 1 to 10.

### AlkylBenzene Oxidation

The oxidation step in the present process is effected by contacting the mixed cumene/butylbenzene feedstock such as described above with an oxygen-containing gas, such as air, in the presence of a catalyst comprising a cyclic imide of the general formula (I):

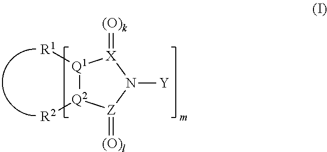

wherein each of R¹ and R² is independently selected from hydrocarbyl and substituted hydrocarbyl radicals having 1 to 20 carbon atoms, or the groups SO₃H, NH₂, OH and NO₂, or the atoms H, F, Cl, Br and I provided that R¹ and R² can be linked to one another via a covalent bond; each of Q¹ and Q² is independently selected from C, CH, N, and CR³; each of X and Z is independently selected from C, S, CH₂, N, P and elements of Group 4 of the Periodic Table; Y is O or OH; k is 0, 1, or 2; l is 0, 1, or 2; m is 1 to 3, and R³ can be any of the entities (radicals, groups, or atoms) listed for R¹. The terms “group”, “radical”, and “substituent” are used interchangeably in this document. For purposes of this disclosure, “hydrocarbyl radical” is defined to be a radical, which contains hydrogen atoms and up to 20 carbon atoms and which may be linear, branched, or cyclic, and when cyclic, aromatic or non-aromatic. “Substituted hydrocarbyl radicals” are radicals in which at least one hydrogen atom in a hydrocarbyl radical has been substituted with at least one functional group or where at least one non-hydrocarbon atom or group has been inserted within the hydrocarbyl radical. Conveniently, each of R¹ and R² is independently selected from aliphatic alkoxy or aromatic alkoxy radicals, carboxyl radicals, alkoxy-carbonyl radicals and hydrocarbon radicals, each of which radicals has 1 to 20 carbon atoms.

Generally, the cyclic imide employed as the oxidation catalyst obeys the general formula

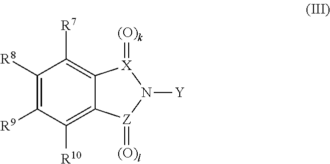

wherein each of R⁷, R⁸, R⁹, and R¹⁰ is independently selected from hydrocarbyl and substituted hydrocarbyl radicals having 1 to 20 carbon atoms, or the groups SO₃H, NH₂, OH and NO₂, or the atoms H, F, Cl, Br and I; each of X and Z is independently selected from C, S, CH₂, N, P and elements of Group 4 of the Periodic Table; Y is O or OH; k is 0, 1, or 2, and l is 0, 1, or 2. Conveniently, each of R⁷, R⁸, R⁹, and R¹⁰ is independently selected from aliphatic alkoxy or aromatic alkoxy radicals, carboxyl radicals, alkoxy-carbonyl radicals and hydrocarbon radicals, each of which radicals has 1 to 20 carbon atoms.

In one practical embodiment, the cyclic imide catalyst comprises N-hydroxyphthalimide (NHPI).

Suitable conditions for the oxidation step include a temperature between about 70° C. and about 200° C., such as about 90° C. to about 130° C. and/or a pressure of about 0.5 to about 20 atmospheres (50 to 2000 kPa). The oxidation reaction is conveniently conducted in a catalytic distillation unit and the per-pass conversion is preferably kept below 50%, to minimize the formation of byproducts.

The oxidation step converts the cumene and sec-butylbenzene in the alkylbenzene mixture to their respective hydroperoxides. However, the oxidation process also tends to generate water and organic acids (e.g., acetic or formic acid) as by-products, which can hydrolyse the catalyst and also lead to decomposition of the hydroperoxide species. Thus, in one embodiment, the conditions employed in the oxidation step, particularly the pressure and oxygen concentration, are controlled so as to maintain the concentration of water and organic acids in the reaction medium below 50 ppm. Such conditions typically include conducting the oxidation at relatively low pressure, such as below 300 kPa, for example between about 100 kPa and about 200 kPa. Moreover, although the oxidation can be conducted over a broad oxygen concentration range between 0.1 and 100%, it is preferred to operate at relatively low oxygen concentration, such as no more than 21 volume %, for example from about 0.1 to about 21 volume %, generally from about 1 to about 10 volume %, oxygen in the oxygen-containing gas. In addition, maintaining the desired low levels of water and organic acids may be facilitated by passing a stripping gas through the reaction medium during the oxidation step. In one embodiment, the stripping gas is the same as the oxygen-containing gas. In another embodiment, the stripping gas is different from the oxygen-containing gas and is inert to the reaction medium and the cyclic imide catalyst. Suitable stripping gases include inert gases, such as helium and argon.

An additional advantage of operating the oxidation process at low pressure and low oxygen concentration and by stripping water and organic acids from the reaction medium is that light hydroperoxide (e.g., ethyl or methyl hydroperoxide), light ketones (e.g., methyl ethyl ketone), light aldehydes (e.g., acetaldehyde) and light alcohols (e.g., ethanol) are removed from the reaction products as they are formed. Thus light hydroperoxides are hazardous and pose a safety concern if their concentration in the liquid product becomes too high. Also, light hydroperoxides, alcohols, aldehydes and ketones are precursors for the formation of organic acids and water so that removing these species from the oxidation medium improves the oxidation reaction rate and selectivity and the stability of the cyclic imide catalyst. In fact, data show that when conducting oxidation of sec-butylbenzene with NHPI at 100 psig (790 kPa), more than 90 mol % of these light species and water remain in the reactor, whereas at atmospheric pressure, more than 95 mol % of these species are removed from the oxidation reactor.

### Oxidation Product

The product of the oxidation process is a mixture of cumene and sec-butylbenzene hydroperoxides, which can be then be converted by acid cleavage to phenol and a mixture of acetone and methyl ethyl ketone.

The cleavage reaction is conveniently affected by contacting the hydroperoxide with a catalyst in the liquid phase at a temperature of about 20° C. to about 150° C., such as about 40° C. to about 120° C., and/or a pressure of about 50 to about 2500 kPa, such as about 100 to about 1000 kPa and/or a liquid hourly space velocity (LHSV) based on the hydroperoxide of about 0.1 to about 1000 hr⁻¹, preferably about 1 to about 50 hr⁻¹. The hydroperoxide is preferably diluted in an organic solvent inert to the cleavage reaction, such as methyl ethyl ketone, phenol, cyclohexylbenzene, cyclohexanone and sec-butylbenzene, to assist in heat removal. The cleavage reaction is conveniently conducted in a catalytic distillation unit.

The catalyst employed in the cleavage step can be a homogeneous catalyst or a heterogeneous catalyst.

Suitable homogeneous cleavage catalysts include sulfuric acid, perchloric acid, phosphoric acid, hydrochloric acid and p-toluenesulfonic acid. Ferric chloride, boron trifluoride, sulfur dioxide and sulfur trioxide are also effective homogeneous cleavage catalysts. The preferred homogeneous cleavage catalyst is sulfuric acid.

A suitable heterogeneous catalyst for use in the cleavage of sec-butylbenzene hydroperoxide includes smectite clay, such as an acidic montmorillonite silica-alumina clay, as described in U.S. Pat. No. 4,870,217 (Texaco), the entire disclosure of which is incorporated herein by reference.

The invention will now be more particularly described with reference to the following non-limiting Examples.

### Example 1

### Effect of Tert-Butylbenzene on Uncatalyzed Oxidation of Cumene/Sec-Butylbenzene Mixture

An alkylbenzene mixture consisting of 20 wt % cumene and 80 wt % sec-butylbenzene was combined with varying amounts of tert-butylbenzene to produce three different oxidation feedstocks containing (a) 0 wt %, (b) 5 wt % and (c) 20 wt % of tert-butylbenzene. Each feedstock was subjected to the following oxidation procedure.

150 gms of the feedstock were weighed into a Parr reactor fitted with a stirrer, thermocouple, gas inlet, sampling port and a condenser containing a DeanStark trap for water removal. The reactor and contents were stirred at 700 rpm and sparged with nitrogen at a flow rate of 250 cc/minute for 5 minutes. The reactor was then pressurized with nitrogen to 40 psig (380 kPa) and, while maintaining a nitrogen sparge, the reactor was heated to 130° C. When the reaction temperature was reached, the gas was switched from nitrogen to air and the reactor was sparged with air at 250 cc/minute for 6 hours. Samples were taken hourly. After 6 hours, the gas was switched back to nitrogen and the heat was turned off. When the reactor had cooled, it was depressurized and the contents removed.

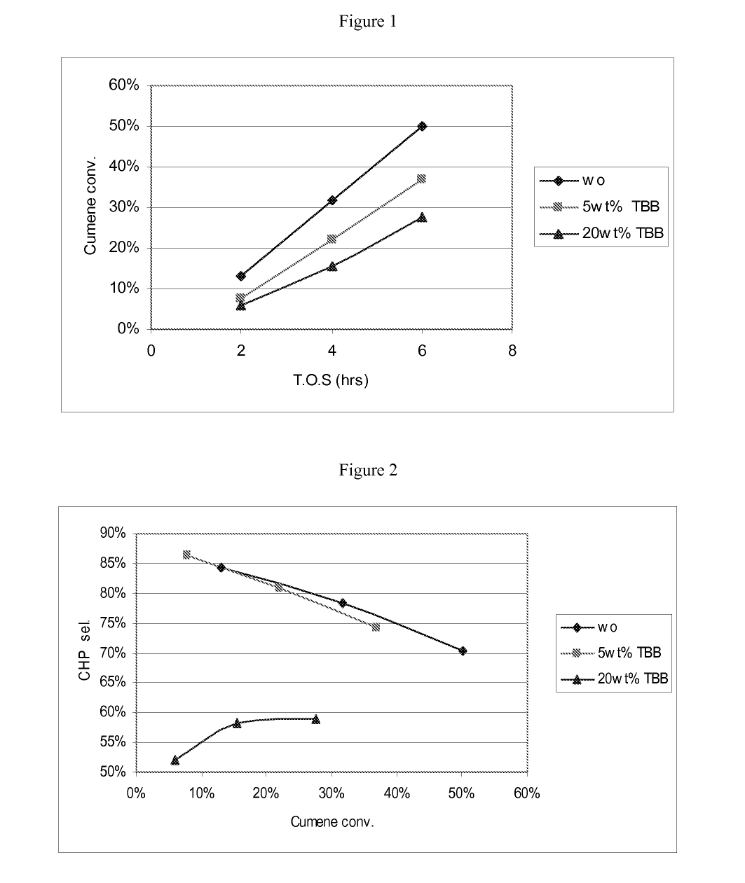

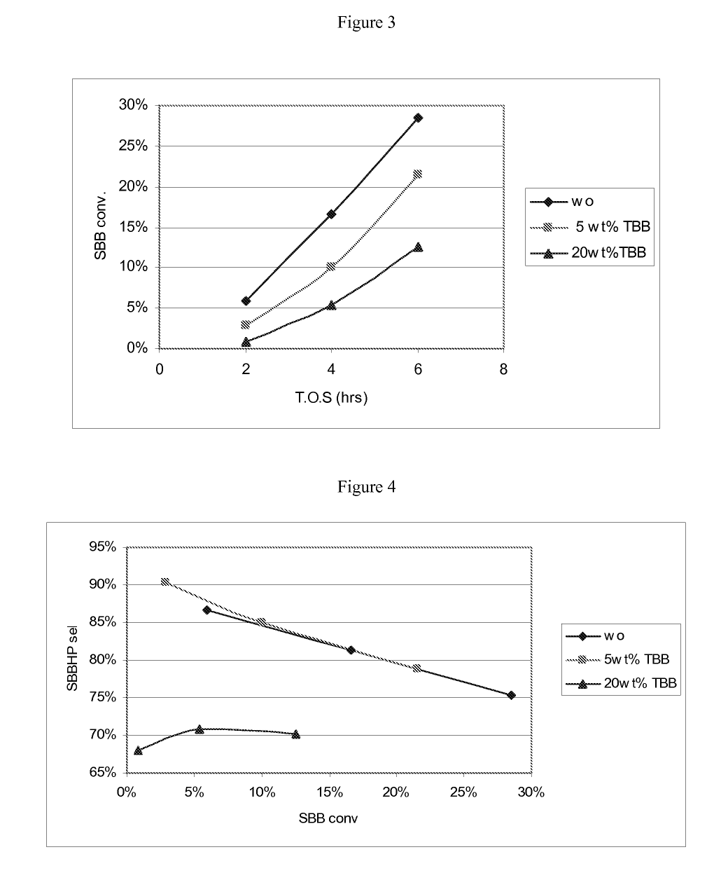

The conversion against time on stream and the hydroperoxide selectivity against conversion for the cumene and sec-butylbenzene components of each feedstock were measured and the results are shown in FIGS. 1 to 4. It will be seen from FIGS. 1 and 3 that the addition of 5 wt %, and especially 20 wt %, of tert-butylbenzene significantly reduced the level of conversion of both the cumene and sec-butylbenzene. Moreover, although the addition of 5 wt % tert-butylbenzene had little effect on the hydroperoxide selectivity, the addition of 20 wt % tert-butylbenzene drastically reduced the selectivity to both cumene hydroperoxide and sec-butylbenzene hydroperoxide (FIGS. 2 and 4).

### Example 2

### Effect of Iso-Butylbenzene on Uncatalyzed Oxidation of Cumene/Sec-Butylbenzene Mixture

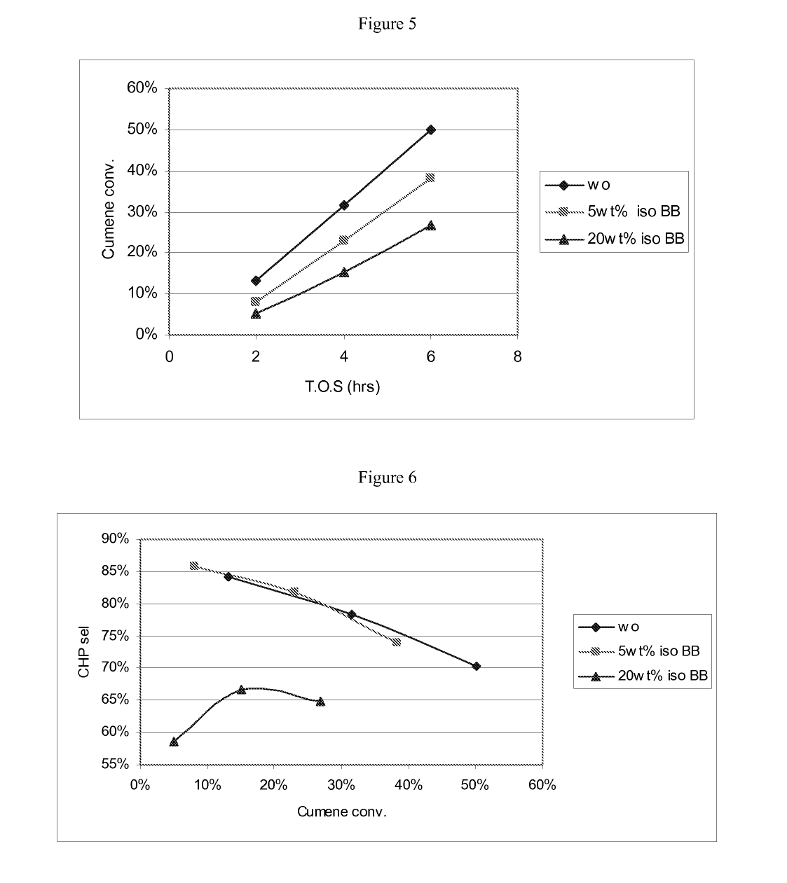

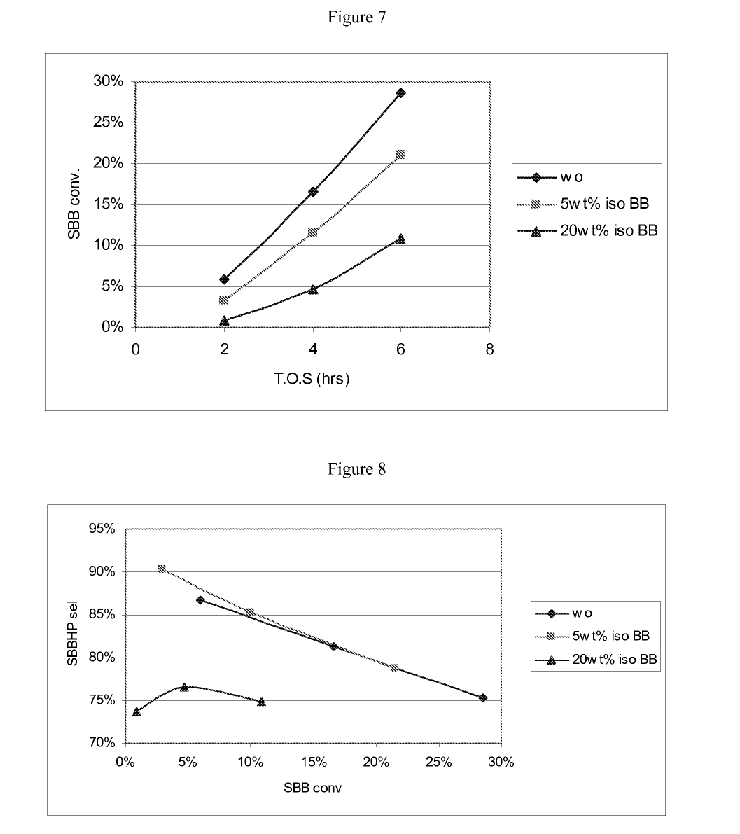

The procedure of Example 1 was repeated but with the alkylbenzene mixture being combined with varying amounts of iso-butylbenzene to produce oxidation feedstocks containing (a) 0 wt %, (b) 5 wt % and (c) 20 wt % of iso-butylbenzene. The results are shown in FIGS. 5 to 8. Again, the addition of 5 wt %, and especially 20 wt %, of iso-butylbenzene significantly reduced the level of conversion of both the cumene and sec-butylbenzene (FIGS. 5 and 7). In addition, although the addition of 5 wt % iso-butylbenzene had little effect on the hydroperoxide selectivity, the addition of 20 wt % iso-butylbenzene drastically reduced the selectivity to both cumene hydroperoxide and sec-butylbenzene hydroperoxide (FIGS. 6 and 8).

### Example 3

### Effect of Tert-Butylbenzene on NHPI Catalyzed Oxidation of Cumene/Sec-Butylbenzene Mixture

An alkylbenzene mixture consisting of 20 wt % cumene and 80 wt % sec-butylbenzene was combined with varying amounts of tert-butylbenzene to produce two different oxidation feedstocks containing (a) 0 wt % and (b) 5 wt % of tert-butylbenzene. Each feedstock was subjected to the following oxidation procedure.

150 gms of the feedstock were weighed with 0.1 wt % of N-hydroxyphthalimide (NHPI) into a Parr reactor fitted with a stirrer, thermocouple, gas inlet, sampling port and a condenser containing a DeanStark trap for water removal. The reactor and contents were stirred at 700 rpm and sparged with nitrogen at a flow rate of 250 cc/minute for 5 minutes. The reactor was then pressurized with nitrogen to 40 psig (380 kPa) and, while maintaining a nitrogen sparge, the reactor was heated to 130° C. When the reaction temperature was reached, the gas was switched from nitrogen to air and the reactor was sparged with air at 250 cc/minute for 4 hours. Samples were taken hourly. After 4 hours, the gas was switched back to nitrogen and the heat was turned off. When the reactor had cooled, it was depressurized and the contents removed.

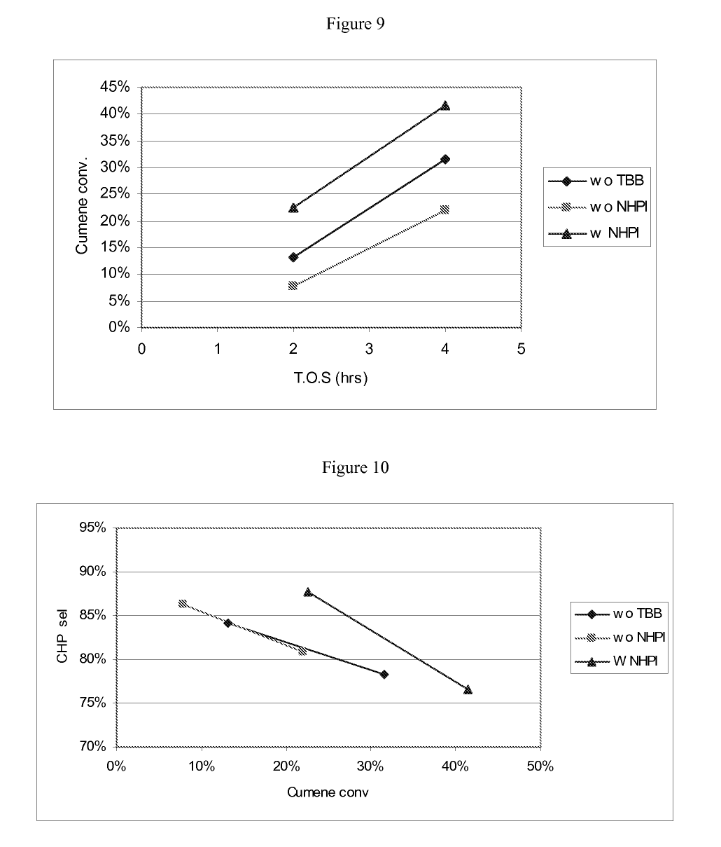

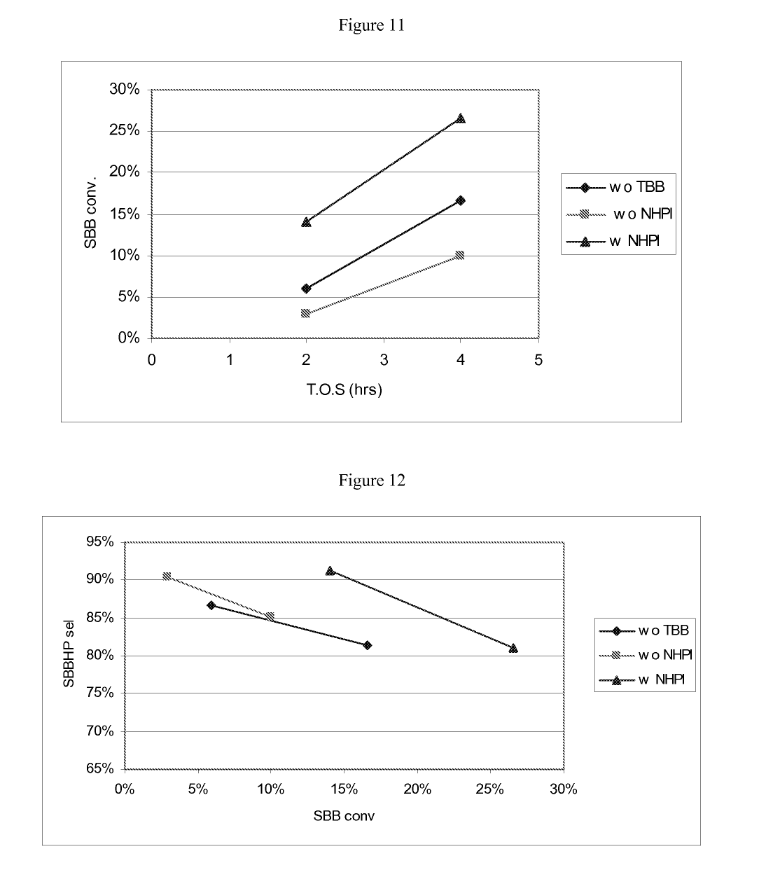

The conversion against time on stream and the hydroperoxide selectivity against conversion for the cumene and sec-butylbenzene components of each feedstock were measured. The results are plotted in FIGS. 9 to 12, which also show the results obtained with the base feedstock (no added tert-butylbenzene) in the absence of the NHPI catalyst. It will be seen from FIGS. 9 and 11 that, in the absence of tert-butylbenzene, the addition of NHPI catalyst improved the level of conversion of both the cumene and sec-butylbenzene, but that this improvement was significantly enhanced by the addition of 5 wt % of tert-butylbenzene. Moreover, although the addition of NHPI catalyst had little effect on the hydroperoxide selectivity with the base cumene/sec-butylbenzene feedstock, with the feedstock containing 5 wt % tert-butylbenzene, addition of the NHPI significantly increased the selectivity to sec-butylbenzene hydroperoxide and to a lesser extent to cumene hydroperoxide and (FIGS. 10 and 12).

### Example 4

### Effect of Iso-Butylbenzene on NHPI Catalyzed Oxidation of Cumene/Sec-Butylbenzene Mixture

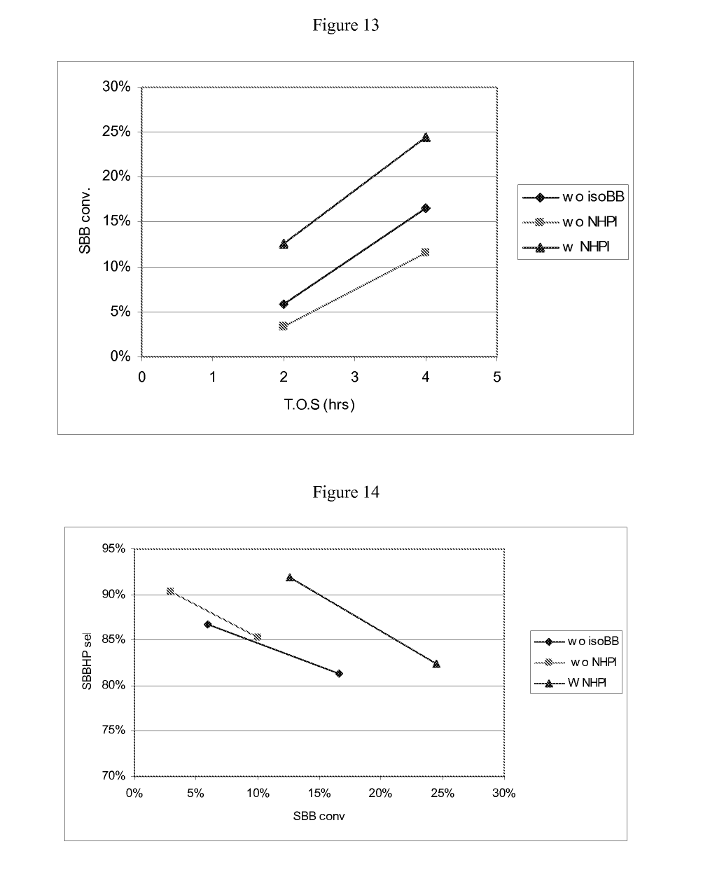

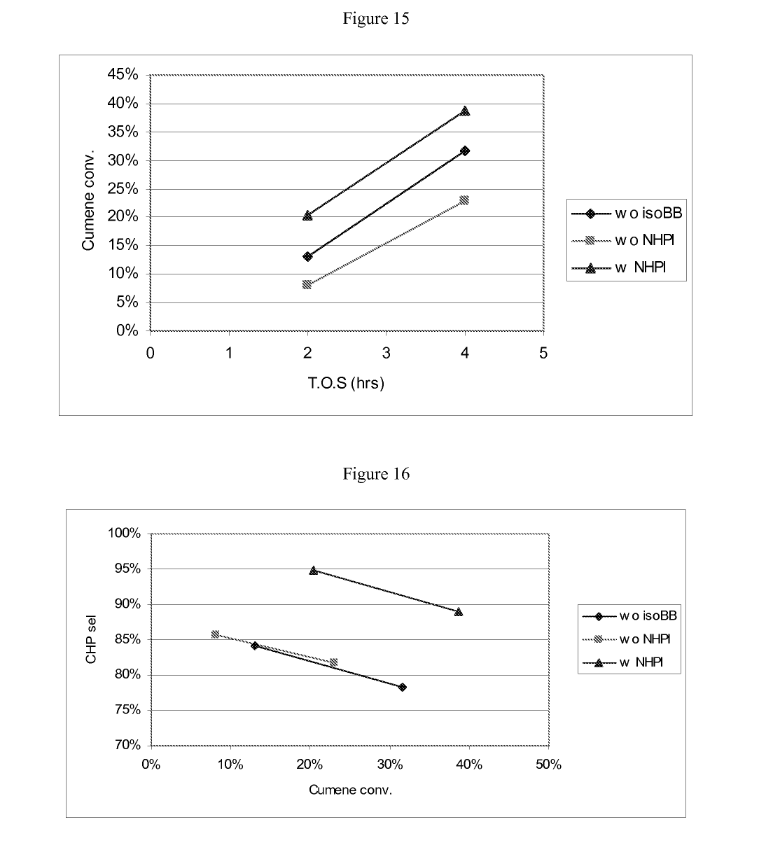

The procedure of Example 3 was repeated but with the alkylbenzene mixture being combined with varying amounts of iso-butylbenzene to produce oxidation feedstocks containing (a) 0 wt % and (b) 5 wt % of iso-butylbenzene. The results are shown in FIGS. 13 to 16, which also show the results obtained with the base feedstock (no added iso-butylbenzene) in the absence of the NHPI catalyst. It will be seen from FIGS. 13 and 15 that, in the absence of iso-butylbenzene, the addition of NHPI catalyst improved the level of conversion of both the sec-butylbenzene and cumene, but that this improvement was significantly enhanced by the addition of 5 wt % of iso-butylbenzene. Moreover, although the addition of NHPI catalyst had little effect on the hydroperoxide selectivity with the base cumene/sec-butylbenzene feedstock, with the feedstock containing 5 wt % iso-butylbenzene, addition of the NHPI significantly increased the selectivity to both sec-butylbenzene hydroperoxide and cumene hydroperoxide and (FIGS. 14 and 16).

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

1. A process for producing alkylbenzene hydroperoxides, the process comprising contacting a feed comprising (i) sec-butylbenzene, (ii) cumene in an amount greater than 10 wt % of the total feed and (iii) at least one of iso-butylbenzene and tert-butylbenzene in an amount up to 20 wt % of the total feed with an oxygen-containing gas in the presence of a catalyst comprising a cyclic imide of the general formula (I): 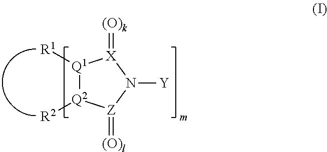
wherein each of R¹ and R² is independently selected from hydrocarbyl and substituted hydrocarbyl radicals having 1 to 20 carbon atoms, or from the groups SO₃H, NH₂, OH, and NO₂ or from the atoms H, F, Cl, Br, and I, provided that R¹ and R² can be linked to one another via a covalent bond;
each of Q¹ and Q² is independently selected from C, CH, N and CR³;
each of X and Z is independently selected from C, S, CH₂, N, P and elements of Group 4 of the Periodic Table;
Y is O or OH;
k is 0, 1, or 2;
l is 0, 1, or 2;
m is 1 to 3; and
R³ can be any of the entities listed for R¹, and wherein said contacting is conducted under conditions to convert said sec-butylbenzene and cumene to the associated hydroperoxides.

2. The process of claim 1, wherein said cyclic imide obeys the general formula (II): 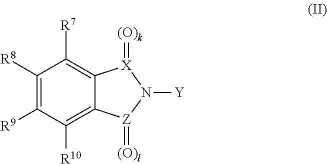
wherein each of R⁷, R⁸, R⁹, and R¹⁰ is independently selected from hydrocarbyl and substituted hydrocarbyl radicals having 1 to 20 carbon atoms, or from the groups SO₃H, NH₂, OH, and NO₂ or from the atoms H, F, Cl, Br, and I,
each of X and Z is independently selected from C, S, CH₂, N, P and elements of Group 4 of the Periodic Table;
Y is O or OH;
k is 0, 1, or 2; and
l is 0, 1, or 2.

3. The process of claim 1, wherein said cyclic imide comprises N-hydroxyphthalimide.

4. The process of claim 1, wherein said feed comprises from 1 wt % to 15 wt % of iso-butylbenzene and/or tert-butylbenzene.

5. The process of claim 1, wherein said feed comprises from 15 wt % to 50 wt % of cumene.

6. The process of claim 1, wherein said contacting is conducted at a temperature of between 90° C. and 150° C.

7. The process of claim 6, wherein said contacting is conducted at a temperature of between 100° C. and 140° C.

8. The process of claim 7, wherein said contacting is conducted at temperature of between 115° C. and 130° C.

9. The process of claim 1, wherein said contacting is conducted at a pressure between 15 kPa and 500 kPa, preferably between 15 kPa and 150 kPa.

10. The process of claim 1, wherein said cyclic imide is present in an amount between 0.05 wt % and 5 wt %, preferably between 0.1 wt % and 1 wt %, of the sec-butylbenzene and cumene in said feed during said contacting.

11. The process of claim 1, and further comprising cleaving the hydroperoxides produced by said contacting to produce phenol, acetone and methyl ethyl ketone.

12. The process of claim 11, wherein said cleaving is conducted in the presence of a catalyst.

13. The process of claim 12, wherein said catalyst is a heterogeneous catalyst.

14. The process of claim 13, wherein said heterogeneous catalyst comprises a smectite clay.

15. The process of claim 11, wherein said cleaving is conducted at a temperature of 40° C. to 120° C. and/or a pressure of 100 to 1000 kPa and/or a liquid hourly space velocity (LHSV) based on the hydroperoxides of 1 to 50 hr⁻¹.

16. The process of claim 11, and further comprising converting the phenol produced by said cleaving to bisphenol A.

17. A process for making phenol, the process comprising:
(i) alkylating a composition comprising: a C3 alkylating agent, a C4 alkylating agent and benzene in the presence of an alkylation catalyst to form sec-butylbenzene and cumene;
(ii) contacting a feed comprising: (a) at least some of the sec-butylbenzene, (b) at least some of the cumene in an amount greater than 10 wt % of the total feed and (c) at least one of iso-butylbenzene and tert-butylbenzene in an amount up to 20 wt % of the total feed with an oxygen-containing gas in the presence of a catalyst comprising a cyclic imide of the general formula (I): 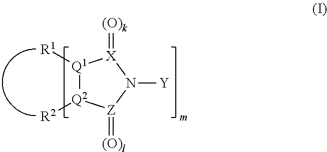
wherein each of R¹ and R² is independently selected from hydrocarbyl and substituted hydrocarbyl radicals having 1 to 20 carbon atoms, or from the groups SO₃H, NH₂, OH, and NO₂ or from the atoms H, F, Cl, Br, and I, provided that R¹ and R² can be linked to one another via a covalent bond;
each of Q¹ and Q² is independently selected from C, CH, N and CR³;
each of X and Z is independently selected from C, S, CH₂, N, P and elements of Group 4 of the Periodic Table;
Y is O or OH;
k is 0, 1, or 2;
l is 0, 1, or 2;
m is 1 to 3; and
R³ can be any of the entities listed for R¹, and wherein said contacting is conducted under conditions to convert said sec-butylbenzene and cumene to the associated hydroperoxides; and
(iii) cleaving at least a portion of the associated hydroperoxides in the presence of a catalyst to form at least some phenol, acetone and methyl ethyl ketone.
